SEQUENCE LISTING (9 sequences)

SEQ ID NO: 1 - protein, 881 aa, Artificial Sequence
  Synthetic (V2E2-GS30 (KiH))
MGWSCIILFLVATATGVHSDIQMTQSPSSLSASVGDRVTITCRASQKIFNGLSWYQQKPGKAPKLLIYHSSTLQSGVPSRFSGSGSGTDFTLTISSLQPEDFATYYCQQVLLYPYTFGQGTKVEIKREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKMQIFVKTLTGKTITLEVEPSDTIENVKAKIQDKEGIPPDQQRLIFAGKQLEDGRTLSDYNIQKESTLHLVLRLRGGGGGGSGGGGSGGGGSGGGGSGGGGSGGGGSMQIFVKTLTGKTITLEVEPSDTIENVKAKIQDKEGIPPDQQRLIFAGKQLEDGRTLSDYNIQKESTLHLVLRLRGGDIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 2 - protein, 777 aa, Artificial Sequence
  Synthetic (V2E2-2 (KiH))
MGWSCIILFLVATATGVHSDIQMTQSPSSLSASVGDRVTITCRASQKIFNGLSWYQQKPGKAPKLLIYHSSTLQSGVPSRFSGSGSGTDFTLTISSLQPEDFATYYCQQVLLYPYTFGQGTKVEIKREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKMQIFVKTLTGKTITLEVEPSDTIENVKAKIQDKEGIPPDQQRLIFAGKQLEDGRTLSDYNIQKESTLHLVLRLRGGGGDIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 3 - protein, 886 aa, Artificial Sequence
  Synthetic (V2E2-GGS30+5 (KiH))
MGWSCIILFLVATATGVHSDIQMTQSPSSLSASVGDRVTITCRASQKIFNGLSWYQQKPGKAPKLLIYHSSTLQSGVPSRFSGSGSGTDFTLTISSLQPEDFATYYCQQVLLYPYTFGQGTKVEIKREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGKMQIFVKTLTGKTITLEVEPSDTIENVKAKIQDKEGIPPDQQRLIFAGKQLEDGRTLSDYNIQKESTLHLVLRLRGGGGGGSGGGGSGGGGSGGGGSGGGGSGGGGSMQIFVKTLTGKTITLEVEPSDTIENVKAKIQDKEGIPPDQQRLIFAGKQLEDGRTLSDYNIQKESTLHLVLRLRGGGGGGSDIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 4 - DNA, 2652 bp, Artificial Sequence
  Synthetic (V2E2-GS30 (KiH))
gccaccatgggctggtcctgcatcatcctgtttctggtggccaccgccaccggcgtgcactctgatatccagatgacccagagccccagcagcctgtctgcctctgtgggcgacagagtgaccatcacctgtcgggccagccagaagatcttcaacggcctgagctggtatcagcagaagcccggcaaggcccccaagctgctgatctaccacagcagcaccctgcagagcggcgtgccaagcagattttctggcagcggcagcggcaccgacttcaccctgacaatcagcagcctgcagcccgaggacttcgccacctactactgccagcaggtcctgctgtacccctacacctttggccagggcaccaaggtggaaatcaagagagagcccaagagctgcgacaagacccacacctgtcccccttgtcctgcccctgaactgctgggaggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagcccagagaaccccaggtgtacacactgccccccagcagagaagagatgaccaagaaccaggtgtccctgtactgcctggtcaagggcttctaccccagcgatatcgccgtggaatgggagagcaacggccagcctgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagatgcagatcttcgtgaaaaccctgaccggcaagaccatcaccctggaagtggaacccagcgacaccatcgagaacgtgaaggccaagatccaggacaaagagggcatcccccccgaccagcagagactgatcttcgccggcaagcagctggaagatggcagaaccctgagcgactacaacatccagaaagagtccaccctgcacctggtgctgcggctgagaggcggaggtggtggtggttctggtggcggaggatctggcggtggtggatctgggggaggtggaagtggtggcggtggtagtggtggtggcggaagcatgcagatttttgtcaagacactgaccgggaaaacaatcacactcgaagtcgagccctccgatacaattgagaatgtgaaagccaaaattcaggacaaagaagggattcctcctgatcagcagcggctgatttttgccggaaaacagctcgaagatggacggaccctgtccgattacaatattcagaaagaaagcaccctccatctggtcctgaggctgcggggaggcgacattcagatgacacagtcccccacctccctgtctgccagcgtgggagatcgcgtgaccattacctgcagagcctcccagtggatcggcaacctgctggattggtatcagcagaaacctggggaggctcctaaactgctgatctattacgccagcttcctgcagtccggcgtgccctctagattttccggcggaggcttcggcacagatttcacactgaccatctcatccctgcagcctgaagattttgccacatattattgccagcaggccaaccctgcccccctgacatttggacagggaacaaaggtcgagatcaagcgcgagcctaagtcctgtgacaagacacacacatgccctccctgcccagccccagaactgctcggtggaccctctgtgtttctgtttccacccaagcctaaggatacactcatgatctccagaacacctgaagtgacatgtgtggtcgtcgacgtgtcacatgaggatccagaagtcaagtttaactggtatgtggatggggtcgaggtgcacaatgccaaaacaaaacctcgggaagaacagtataattccacctatagagtcgtgtctgtgctcaccgtgctccatcaggattggctcaatgggaaagaatacaaatgtaaagtctctaacaaagccctgcccgctcctatcgaaaagacaatctccaaggccaaaggacagcctcgcgagcctcaggtctacaccctgccaccttcccgcgaggaaatgacaaaaaatcaggtgtcactcacctgtctcgtgaaggggttttacccctccgacattgccgtcgagtgggagtccaatggacagcccgagaacaattataagacaacacctcccgtcctggactccgatggatcattttttctgacctccaagctcaccgtcgataagtccagatggcagcagggaaatgtcttttcctgctccgtgatgcatgaagctctccacaatcattacacacagaaaagcctgtccctgtcccccggcaagtga

SEQ ID NO: 5 - DNA, 2340 bp, Artificial Sequence
  Synthetic (V2E2-2 (KiH))
gccaccatgggctggtcctgcatcatcctgtttctggtggccaccgccaccggcgtgcactctgatatccagatgacccagagccccagcagcctgtctgcctctgtgggcgacagagtgaccatcacctgtcgggccagccagaagatcttcaacggcctgagctggtatcagcagaagcccggcaaggcccccaagctgctgatctaccacagcagcaccctgcagagcggcgtgccaagcagattttctggcagcggcagcggcaccgacttcaccctgacaatcagcagcctgcagcccgaggacttcgccacctactactgccagcaggtcctgctgtacccctacacctttggccagggcaccaaggtggaaatcaagagagagcccaagagctgcgacaagacccacacctgtcccccttgtcctgcccctgaactgctgggaggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagcccagagaaccccaggtgtacacactgccccccagcagagaagagatgaccaagaaccaggtgtccctgtactgcctggtcaagggcttctaccccagcgatatcgccgtggaatgggagagcaacggccagcctgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagatgcagatcttcgtgaaaaccctgaccggcaagaccatcaccctggaagtggaacccagcgacaccatcgagaacgtgaaggccaagatccaggacaaagagggcatcccccccgaccagcagagactgatcttcgccggcaagcagctggaagatggcagaaccctgagcgactacaacatccagaaagagtccaccctgcacctggtgctgcggctgagaggcggaggtggtgacattcagatgacacagtcccccacctccctgtctgccagcgtgggagatcgcgtgaccattacctgcagagcctcccagtggatcggcaacctgctggattggtatcagcagaaacctggggaggctcctaaactgctgatctattacgccagcttcctgcagtccggcgtgccctctagattttccggcggaggcttcggcacagatttcacactgaccatctcatccctgcagcctgaagattttgccacatattattgccagcaggccaaccctgcccccctgacatttggacagggaacaaaggtcgagatcaagcgcgagcctaagtcctgtgacaagacacacacatgccctccctgcccagccccagaactgctcggtggaccctctgtgtttctgtttccacccaagcctaaggatacactcatgatctccagaacacctgaagtgacatgtgtggtcgtcgacgtgtcacatgaggatccagaagtcaagtttaactggtatgtggatggggtcgaggtgcacaatgccaaaacaaaacctcgggaagaacagtataattccacctatagagtcgtgtctgtgctcaccgtgctccatcaggattggctcaatgggaaagaatacaaatgtaaagtctctaacaaagccctgcccgctcctatcgaaaagacaatctccaaggccaaaggacagcctcgcgagcctcaggtctacaccctgccaccttcccgcgaggaaatgacaaaaaatcaggtgtcactcacctgtctcgtgaaggggttttacccctccgacattgccgtcgagtgggagtccaatggacagcccgagaacaattataagacaacacctcccgtcctggactccgatggatcattttttctgacctccaagctcaccgtcgataagtccagatggcagcagggaaatgtcttttcctgctccgtgatgcatgaagctctccacaatcattacacacagaaaagcctgtccctgtcccccggcaagtga

SEQ ID NO: 6 - DNA, 2667 bp, Artificial Sequence
  Synthetic (V2E2-GS30+5 (KiH))
gccaccatgggctggtcctgcatcatcctgtttctggtggccaccgccaccggcgtgcactctgatatccagatgacccagagccccagcagcctgtctgcctctgtgggcgacagagtgaccatcacctgtcgggccagccagaagatcttcaacggcctgagctggtatcagcagaagcccggcaaggcccccaagctgctgatctaccacagcagcaccctgcagagcggcgtgccaagcagattttctggcagcggcagcggcaccgacttcaccctgacaatcagcagcctgcagcccgaggacttcgccacctactactgccagcaggtcctgctgtacccctacacctttggccagggcaccaaggtggaaatcaagagagagcccaagagctgcgacaagacccacacctgtcccccttgtcctgcccctgaactgctgggaggccctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccatcagcaaggccaagggccagcccagagaaccccaggtgtacacactgccccccagcagagaagagatgaccaagaaccaggtgtccctgtactgcctggtcaagggcttctaccccagcgatatcgccgtggaatgggagagcaacggccagcctgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagatgcagatcttcgtgaaaaccctgaccggcaagaccatcaccctggaagtggaacccagcgacaccatcgagaacgtgaaggccaagatccaggacaaagagggcatcccccccgaccagcagagactgatcttcgccggcaagcagctggaagatggcagaaccctgagcgactacaacatccagaaagagtccaccctgcacctggtgctgcggctgagaggcggaggtggtggtggttctggtggcggaggatctggcggtggtggatctgggggaggtggaagtggtggcggtggtagtggtggtggcggaagcatgcagatttttgtcaagacactgaccgggaaaacaatcacactcgaagtcgagccctccgatacaattgagaatgtgaaagccaaaattcaggacaaagaagggattcctcctgatcagcagcggctgatttttgccggaaaacagctcgaagatggacggaccctgtccgattacaatattcagaaagaaagcaccctccatctggtcctgaggctgcggggaggcggcggcggcggcagcgacattcagatgacacagtcccccacctccctgtctgccagcgtgggagatcgcgtgaccattacctgcagagcctcccagtggatcggcaacctgctggattggtatcagcagaaacctggggaggctcctaaactgctgatctattacgccagcttcctgcagtccggcgtgccctctagattttccggcggaggcttcggcacagatttcacactgaccatctcatccctgcagcctgaagattttgccacatattattgccagcaggccaaccctgcccccctgacatttggacagggaacaaaggtcgagatcaagcgcgagcctaagtcctgtgacaagacacacacatgccctccctgcccagccccagaactgctcggtggaccctctgtgtttctgtttccacccaagcctaaggatacactcatgatctccagaacacctgaagtgacatgtgtggtcgtcgacgtgtcacatgaggatccagaagtcaagtttaactggtatgtggatggggtcgaggtgcacaatgccaaaacaaaacctcgggaagaacagtataattccacctatagagtcgtgtctgtgctcaccgtgctccatcaggattggctcaatgggaaagaatacaaatgtaaagtctctaacaaagccctgcccgctcctatcgaaaagacaatctccaaggccaaaggacagcctcgcgagcctcaggtctacaccctgccaccttcccgcgaggaaatgacaaaaaatcaggtgtcactcacctgtctcgtgaaggggttttacccctccgacattgccgtcgagtgggagtccaatggacagcccgagaacaattataagacaacacctcccgtcctggactccgatggatcattttttctgacctccaagctcaccgtcgataagtccagatggcagcagggaaatgtcttttcctgctccgtgatgcatgaagctctccacaatcattacacacagaaaagcctgtccctgtcccccggcaagtga

SEQ ID NO: 7 - DNA, 1329 bp, Artificial Sequence
  Synthetic (V2Ub (knob))
aagcttgccaccatgggctggagctgcatcatcctgttcctggtggccaccgccaccggcgtgcacagcgacatccagatgacccagagccccagcagcctgagcgccagcgtgggcgaccgcgtgaccatcacctgccgcgccagccagaagatcttcaacggcctgagctggtaccagcagaagcccggcaaggcccccaagctgctgatctaccacagcagcaccctgcagagcggcgtgcccagccgcttcagcggcagcggcagcggcaccgacttcaccctgaccatcagcagcctgcagcccgaggacttcgccacctactactgccagcaggtgctgctgtacccctacaccttcggccagggcaccaaggtggagatcaagcgcgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgaggagatgaccaagaaccaggtgagcctgtactgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaagatgcagatcttcgtgaagaccctgaccggcaagaccatcaccctggaggtggagcccagcgacaccatcgagaacgtgaaggccaagatccaggacaaggagggcatcccccccgaccagcagcgcctgatcttcgccggcaagcagctggaggacggccgcaccctgagcgactacaacatccagaaggagagcaccctgcacctggtgctgcgcctgcgcggcggcaagtaactcgag

SEQ ID NO: 8 - DNA, 1098 bp, Artificial Sequence
  Synthetic (E2 (hole))
aagcttgccaccatgggctggagctgcatcatcctgttcctggtggccaccgccaccggcgtgcacagcgacatccagatgacccagagccccaccagcctgagcgccagcgtgggcgaccgcgtgaccatcacctgccgcgccagccagtggatcggcaacctgctggactggtaccagcagaagcccggcgaggcccccaagctgctgatctactacgccagcttcctgcagagcggcgtgcccagccgcttcagcggcggcggcttcggcaccgacttcaccctgaccatcagcagcctgcagcccgaggacttcgccacctactactgccagcaggccaaccccgcccccctgaccttcggccagggcaccaaggtggagatcaagcgcgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgaccagcaagctgaccgtggacaagagccgctggcagcagggcaacgtgttcagctgcagcgtgatgcacgaggccctgcacaaccactacacccagaagagcctgagcctgagccccggcaagtaactcgag

SEQ ID NO: 9 - protein, 76 aa, Artificial Sequence [sequence omitted]

(19) United States
(12) Patent Application Publication
(10) Pub. No.: US 2014/0081002 A1
(43) Pub. Date: Mar. 20, 2014

(54) PROTEIN COMPLEX INCLUDING BI-SPECIFIC ANTIBODY

(71) Applicant: Samsung Electronics Co., Ltd., Suwon-si (KR)

(72) Inventors: Jae-il LEE, Yongin-si (KR); Min-kyung KIM, Seoul (KR); Yoon-aa CHOI, Busan (KR)

(21) Appl. No.: 14/030,658

(22) Filed: Sep. 18, 2013

(30) Foreign Application Priority Data
Sep. 18, 2012 (KR) 10-2012-0103607

(51) Int. Cl. C07K 16/46; C07K 16/28; C07K 16/22

(52) U.S. Cl. CPC C07K 16/468; C07K 16/22; C07K 16/2863
USPC 530/387.3; 536/23.4; 435/320.1; 435/328; 435/69.6

(57) ABSTRACT

Provided is a protein complex including a bi-specific antibody, and a method of preparing the protein complex, which can provide a system that simultaneously targets two antigens.

### CROSS-REFERENCE TO RELATED APPLICATIONS

This application claims the benefit of Korean Patent Application No. 10-2012-0103607, filed on Sep. 18, 2012, in the Korean Intellectual Property Office, the entire disclosure of which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 39,655 Byte ASCII (Text) file named “713835_ST25.TXT,” created on Sep. 16, 2013.

### BACKGROUND

1. Field

The present disclosure relates to protein complexes including bi-specific antibodies and a method of preparing the protein complexes.

2. Description of the Related Art

Monoclonal antibodies have been leading a new drug market and developed as a therapeutic agent for a variety of targets. In many cases, however, monoclonal antibodies do not have a satisfactory efficacy and development thereof as a new drug has limitations due to their high manufacturing costs. To address these problems, research into bi-specific antibodies has been continuously conducted since the middle 1980s. In spite of so much effort, a leading technology for producing bi-specific antibodies has not yet been reported.

A preexisting method of producing bi-specific antibodies has disadvantages: difficulties in mass producing bi-specific antibodies and difficulties in commercialization thereof due to low efficacy and side effects. Recently, thanks to advanced antibody engineering, competitive new antibody platforms have emerged, but the antibody platforms are still in a verification stage.

Therefore, there is still a need to develop a protein complex that is specific to at least two new heteroantigens.

### SUMMARY

Provided are protein complexes for the production of bi-specific antibodies. The protein complex can comprise a first polypeptide comprising a first antigen-binding site at the N-terminus thereof; a second polypeptide comprising a second antigen-binding site at the N-terminus thereof; and a linker that links the first and second polypeptides to each other, wherein the first polypeptide and second polypeptide each comprises a domain including at least one knob or hole on a region other than the first or second antigen-binding site, wherein, if the first polypeptide comprises at least one knob, then the second polypeptide comprises a domain including at least one hole on a region other than the second antigen-binding site, wherein, if the first polypeptide comprises at least one hole, then the second polypeptide comprises a domain including at least one knob on a region other than the second antigen-binding site, wherein the knob and the hole bind to each other so that the first and second polypeptides form dimers, wherein a first tag and a second tag are bound to both termini of the linker, and wherein the first tag is linked to the C-terminus of the first polypeptide, the second tag is linked to the N-terminus of the second polypeptide, and the first tag and the second tag each comprise a cleavable amino acid sequence.

In another embodiment, the protein complex can comprise a first polypeptide comprising a first antigen-binding site at the N-terminus thereof; a second polypeptide comprising a second antigen-binding site at the N-terminus thereof; and a linker that links the first and second polypeptides to each other, wherein the first and second polypeptides each comprises a domain including at least one knob or hole on a region other than the first or second antigen-binding site, wherein, if the first polypeptide comprises at least one knob, then the second polypeptide comprises a domain including at least one hole on a region other than the second antigen-binding site, wherein, if the first polypeptide comprises at least one hole, then the second polypeptide comprises a domain including at least one knob on a region other than the second antigen-binding site, wherein the knob and the hole can bind to each other so that the first and second polypeptides form dimers, wherein a tag is bound to a terminus of the linker, and wherein the tag is linked to the C-terminus of the first polypeptide or the N-terminus of the second polypeptide and comprises a cleavable amino acid sequence.

Provided are polynucleotides that encode the protein complexes.

Provided are recombinant vectors that include the polynucleotides.

Provided are host cells that include the recombinant vectors.

Provided are methods of preparing bi-specific antibodies by using the protein complexes.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram illustrating a protein complex including a first and second polypeptides and a method of preparing bi-specific antibodies, according to an embodiment;

FIG. 2 is a schematic diagram illustrating a protein complex including a first and second polypeptides and a method of preparing bi-specific antibodies, according to an embodiment;

FIG. 3 illustrates the structure of a protein complex according to an embodiment;

FIG. 4 illustrates sodium dodecyl sulfate polyacrylamide gel electrophoresis (SDS-PAGE) results of a protein complex according to an embodiment that is treated (+) or is not treated (−) with β-mercaptoethanol; and

FIG. 5 is a sensorgram illustrating bi-specific antigen-antibody reaction effects of a protein complex according to an embodiment.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

According to an aspect of the present invention, a protein complex comprises, consists essentially of, or consists of a first polypeptide including a first antigen-binding site at the N-terminus thereof; a second polypeptide including a second antigen-binding site at the N-terminus thereof; and a linker that links the first and second polypeptides to each other, wherein the first and second polypeptides include a domain including at least one knob or hole on a region other than the first or second antigen-binding site, wherein, if the first polypeptide comprises at least one knob, then the second polypeptide comprises a domain including at least one hole on a region other than the second antigen-binding site, wherein, if the first polypeptide comprises at least one hole, then the second polypeptide comprises a domain including at least one knob on a region other than the second antigen-binding site, wherein the knob and the hole can bind each other so that the first and second polypeptides form dimers, wherein a first tag and a second tag are bound at both termini of the linker, wherein the first tag is linked to the C-terminus of the first polypeptide, the second tag is linked to the N-terminus of the second polypeptide, and the first tag and the second tag each include a cleavable amino acid sequence.

According to another aspect of the present invention, a protein complex comprises, consists essentially of, or consists of a first polypeptide including a first antigen-binding site at the N-terminus thereof; a second polypeptide including a second antigen-binding site at the N-terminus thereof; and a linker that links the first and second polypeptides to each other, wherein the first and second polypeptides include a domain including at least one knob or hole on a region other than the first or second antigen-binding site, wherein, if the first polypeptide comprises at least one knob, then the second polypeptide comprises a domain including at least one hole on a region other than the second antigen-binding site, wherein, if the first polypeptide comprises at least one hole, then the second polypeptide comprises a domain including at least one knob on a region other than the second antigen-binding site, wherein the knob and the hole can bind to each other so that the first and second polypeptides form dimers, wherein a tag is bound at a terminus of the linker, wherein the tag is linked to the C-terminus of the first polypeptide or the N-terminus of the second polypeptide and includes a cleavable amino acid sequence.

The term “antigen binding site” used herein collectively refers to sites to which an antigen or an epitope binds in immunoglobulin molecules, and the antigen binding site may include a complementarity determining region (CDR). The CDR refers to an amino acid sequence found in the variable region of a heavy chain or a light chain of an immunoglobulin. The heavy chain and the light chain may include three CDRs (e.g., CDRH1, CDRH2, CDRH3) and three CDRs (CDRL1, CDRL2, CDRL3), respectively. The CDR may provide a major contact residue in antigen or epitope-antibody binding. The term “heavy chain” used herein is understood to include a full-length heavy chain including a variable region (V_{H}) having amino acid sequences that determine specificity for antigens and a constant region having three constant domains (C_{H1}, C_{H2}, and C_{H3}), and fragments thereof. In addition, the term “light chain” used herein is understood to include a full-length light chain including a variable region (V_{L}) having amino acid sequences that determine specificity for antigens and a constant region (C_{L}), and fragments thereof.

The protein complex may have antigen-binding sites that are identical to or different from each other. In other words, the first and second antigen-binding sites, which are antigen binding sites of the first and second polypeptides, may be antigen-binding sites of the same or different antigens. Also, even in the case of the same antigens, the first and second polypeptides may be interpreted to include antigen binding sites capable of binding to different epitopes. Examples of the antigens capable of binding to the antigen binding sites may be selected from, but not limited to, the group consisting of DLL4, VEGFR2, Notch1, Notch2, Notch3, Notch4, Notch(pan), JAG1, JAG2, DLL(pan), JAG(pan), ERBB(pan), c-Met, IGF-1R, PDGFR, Patched, Hedgehog family polypeptides, Hedgehog(pan), WNT family polypeptides, WNT(pan), FZD1, FZD2, FZD3, FZD4, FZD5, FZD6, FZD7, FZD8, FZD9, FZD10, FZD(pan), LRP5, LRP6, CD20, IL-17, CD86, Muc16, PSCA, CD44, c-Kit, DDR1, DDR2, RSPO1, RSPO2, RSPO3, RSPO4, RSPO(pan), BMP family polypeptides, BMP(pan), BMPR1a, BMPR1b, and combinations thereof. Also, examples of the antigens capable of binding to the antigen binding sites may be selected from, but not limited to, the group consisting of Epithelial cell adhesion molecule (EpCAM), tumor-associated glycoprotein-72 (TAG-72), tumor-associated antigen CA 125, Prostate specific membrane antigen (PSMA), High molecular weight melanoma-associated antigen (HMW-MAA), tumor-associated antigen expressing Lewis Y related carbohydrate, Carcinoembryonic antigen (CEA), Carcinoembryonic antigen-related cell adhesion molecule 5 (CEACAM5), Human milk fat globule polymorphic epithelial mucin (HMFG PEM), mucin MUC1, MUC18 and cytokeratin tumor-associated antigen, bacterial antigens, viral antigens, allergens, fluorescein, lysozyme, toll-like receptor 9, erythropoietin, cluster of differentiation 2 (CD2), CD3, CD3E, CD4, CD11, CD11a, CD14, CD18, CD19, CD20, CD22, CD23, CD25, CD28, CD29, CD30, CD33 (p67 protein), CD38, CD40, CD40L, CD52, CD54, CD56, CD80, CD147, GD3, Interleukin 1 (IL-1), IL-1R, IL-2, IL-2R, IL-4, IL-5, IL-6, IL-6R, IL-8, IL-12, IL-15, IL-18, IL-23, interferon alpha, interferon beta, interferon gamma; tumour necrotic factor-alpha (TNF-alpha), TNF-beta2, TNF-alpha, TNF-alphabeta, TNF-R1, TNF-R11, Fas ligand (FasL) (CD95L), CD27L, CD3OL, 4-1BBL, TNF-Related Apoptosis-Inducing Ligand (TRAIL), Receptor activator of nuclear factor kappa-B ligand (RANKL), TNF-related weak inducer of apoptosis (TWEAK), Antibodies against a proliferating inducing ligand (APRIL), B-cell activating factor (BAFF), LIGHT, VEG1, OX4OL, TRAIL Receptor-1, A1 Adenosine Receptor, Lymphotoxin Beta Receptor, TACI, BAFF-R, EPO; LFA-3, ICAM-1, ICAM-3, integrin beta1, integrin beta2, integrin alpha4/beta7, integrin alpha2, integrin alpha3, integrin alpha4, integrin alpha5, integrin alpha6, integrin alphav, alphaVbeta3 integrin, FGFR-3, Keratinocyte Growth Factor, VLA-1, VLA-4, L-selectin, anti-Id, E-selectin, HLA, HLADR, CTLA-4, T cell receptor, B7-1, B7-2, VNRintegrin, TGFbeta1, TGFbeta2, eotaxin1, BLyS (B-lymphocyte Stimulator), complement C5, IgE, factor VII, CD64, CBL, NCA 90, EGFR (ErbB-1), Her2/neu (ErbB-2), Her3 (ErbB-3), Her4 (ErbB4), Tissue Factor, VEGF, VEGFR, endothelin receptor, VLA-4, carbohydrate such as blood group antigen and carbohydrate associated therewith, Galili-Glycosylation, Gastrin, Gastrin receptors, tumor associated carbohydrate, Hapten NP-cap or NIP-cap, T cell receptor alpha/beta, E-selectin, digoxin, placental alkaline phosphatase (PLAP) and testicular PLAP-like alkaline phosphatase, transferrin receptor, Heparanase I, human cardiac myosin, Glycoprotein IIb/IIIa (GPIIb/IIIa), human cytomegalovirus (HCMV) gH envelope glycoprotein, HIV gp120, HCMV, respiratory syncital virus RSV F, RSVF Fgp, VNR integrin, Hep B gp120, CMV, gpIIbIIIa, HIV IIIB gp120 V3 loop, respiratory syncytial virus (RSV) Fgp, Herpes simplex virus (HSV) gD glycoprotein, HSV gB glycoprotein, HCMV gB envelope glycoprotein, Clostridium perfringens toxin, and fragments thereof.

The first and second polypeptides may be a polypeptide comprising, consisting essentially of, or consisting of an antigen-binding site and a Fc domain, wherein the antigen-binding site is selected from the group consisting of a single-domain antibody, Fab, Fab′, and scFv.

In an embodiment, the protein complex may include a linker that links the first and second polypeptides to each other. The linker may be a peptide linker. The peptide linker may be one of various linkers known in the art, for example, a linker may be a plurality of amino acids. For example, the linker may be a polypeptide of 1 to 100 amino acids, for example, 2 to 50 amino acids (e.g., 5, 10, 15, 20, 25, 30, 35, 40, or 45 amino acids, as well as ranges thereof).

The peptide linker allows the at least two polypeptides to be sufficiently spaced apart from each other so that each polypeptide can be folded in a secondary or tertiary structure which is suitable for appropriate function of the polypeptides. For example, the peptide linker may include Gly, Asn and Ser residues, and may also include neutral amino acids such as Thr and Ala. Appropriate amino acid sequences for the peptide linker are well known in the art. The length of the linker may be appropriately adjusted as long as it does not affect the function of the polypeptides.

The first and second polypeptides include a domain including at least one knob or hole on a region other than the first or second antigen-binding site, wherein a knob and a hole can bind to each other so that the first and second polypeptides form dimers.

Generally, dimers are formed in a cell as Fc regions of two heavy chains are bound to each other during the production of antibodies in a cell. Particularly, during the production of bi-specific antibodies, a production rate of the bi-specific antibody may be lowered as homodimer and heterodimer formation are similarly probable when a general (conventional) method of producing antibodies is used.

To increase the possibility of forming heterodimers, the first and second polypeptides each comprises a domain including at least one knob or hole on a region other than the first or second antigen-binding site, wherein a knob and a hole can bind to each other so that the first and second polypeptides form dimers to increase the production rate of the bi-specific antibody.

The term “knob” or “hole” indicates a sequence formed of at least one amino acid located at a region other than the antigen-binding sites of the first and second polypeptides. In a tertiary structure of a protein, the knob may include at least one amino acid that may form a proturberance structure, and the hole may include at least one amino acid that may form a cavity structure (see, e.g., Merchant et al., Nat. Biotechnol., 16: 677 (1998)).

Thus, when the first and second polypeptides are adjacent to each other, a domain including a knob and a domain including a hole may bound to each other, and thus a dimer may be formed. The knob or hole may be introduced by substituting a base sequence in a polynucleotide which encodes the region other than the antigen-binding site of the first or second polypeptide.

If the first polypeptide comprises at least one knob, then the second polypeptide comprises a domain including at least one hole on a region other than the second antigen-binding site. Similarly, if the first polypeptide comprises at least one hole, then the second polypeptide comprises a domain including at least one knob on a region other than the second antigen-binding site. The amino acid for the knob may include an amino acid selected from the group consisting of Arg, Phe, Tyr, and Trp, and the amino acid for the hole may be an amino acid selected from the group consisting of Ala, Ser, Thr, and Val. The knob or hole may have any combination of amino acids as long as the amino acids are a pair of amino acid sequences of which amino acid residues may bond to each other in a domain including the knob and hole, and the amino acid may include a natural or non-natural amino acid. The pair of the amino acid sequences may be, for example, Arg/Ala, Phe/Ser, Tyr/Thr, or Trp/Val, but is not limited thereto.

The region other than the antigen-binding site of the first or second polypeptide may be a CH3 domain of the Fc region of the antibody. As stated above, when a domain including at least one knob or hole, wherein a knob and a hole may be bound to each other, is included in the region other than the antigen-binding site of the first or second polypeptide, a production rate of the dimers may be increased by increasing a molecular force between the first and second polypeptides.

In an embodiment, a tag may be bound to at least one terminus of the linker. In addition, the tag is linked to one of the termini of the polypeptides and may include a cleavable amino acid sequence.

The term “tag” used herein means protein or polypeptide as a medium to link between polypeptides that are different from each other. In an embodiment, the tag may be attached to N-terminus or C-terminus of the polypeptide.

In an embodiment, the tag may include an in vitro or in vivo cleavable amino acid sequence. The in vitro or in vivo cleavage process may be performed by protease. For example, the tag may be selected from the group consisting of ubiquitin, ubiquitin-like protein, a TEV cleavage peptide, and a furin cleavage peptide, but is not limited thereto (see, e.g., Kapust et al., Biochem. Biophys. Res. Commun., 294 (5): 949-955 (2002)).

Ubiquitin (Ub) is the most conserved protein found in nature that consists of 76 amino acids and is a water-soluble protein exhibiting perfect homology among evolutionally various species, such as insects, trout, and humans. In addition, ubiquitin is known to be protein that is stable against pH changes, is not easily denatured at high temperatures, and is stable with respect to protease. Therefore, ubiquitin may improve an insolubility of the protein complex and may be easily cleaved in vitro or in vivo.

The ubiquitin or the ubiquitin-like protein may be selected from the group consisting of wild-type ubiquitin, a wild-type ubiquitin-like protein, mutant ubiquitin, and a mutant ubiquitin-like protein. The mutant ubiquitin is obtained by changing the amino acid sequence of wild-type ubiquitin (SEQ ID NO: 9) into another amino acid sequence. For example, a mutant ubiquitin may be prepared by substituting Lys of wild-type ubiquitin with Arg or by substituting the RGG residues of the C-terminus of wild-type ubiquitin with RGA residues. According to an embodiment, in mutant ubiquitins prepared by substituting Lys of wild-type ubiquitin with Arg, Lys residues that exist at the 6^{th}, 11^{th}, 27^{th}, 29^{th}, 33^{rd}, 48^{th}, and 63^{rd} amino acid positions may be substituted with Arg independently or in any combination. The ubiquitin-like protein is a protein having properties that are similar to those of ubiquitin. Examples of the ubiquitin-like protein may be selected from, but not limited to, the group consisting of Nedd8 (GenBank Accession No.: NP_{—}006147.1), SUMO-1 (GenBank Accession No.: NP_{—}00100578.1), SUMO-2 (GenBank Accession No.: NP_{—}001005849.1), NUB1 (GenBank Accession No.: NP_{—}001230280.1), PIC1 (GenBank Accession No.: NP_{—}001005782.1), UBL3 (GenBank Accession No.: NP_{—}009037.1), UBL5 (GenBank Accession No.: NP_{—}001041706.1), and ISG15 (GenBank Accession No.: NP_{—}005092.10).

In an embodiment, the ubiquitin or ubiquitin-like protein has an amino acid sequence at the C-terminus which can be cleaved in vitro or in vivo by a protease. The amino acid sequence that is cleaved by a protease may be identified by searching a database that is known in the art. For example, a protease and an amino acid sequence that is cleaved by the protease which are searched at www.expasy.org/tools/peptidecutter/peptidecutter_enzymes.html may be used. When the protein complex includes the cleavable amino acid sequence, the tag included in the protein complex is cleaved in vitro or in vivo, whereby at least two fusion proteins may function as a protein complex having a bi-specific or multi-specific antigen binding sites.

The protein complex may be selected from the group consisting of polypeptides of the amino acid sequences of SEQ ID NO: 1 to SEQ ID NO: 3.

According to another aspect of the present invention, a polynucleotide is provided, wherein the polynucleotide encodes a protein complex including a first polypeptide including a first antigen-binding site at the N-terminus thereof; a second polypeptide including a second antigen-binding site at the N-terminus thereof; and a linker that links the first and second polypeptides to each other, wherein the first and second polypeptides include a domain including at least one knob or hole on a region other than the first or second antigen-binding site, wherein, if the first polypeptide comprises at least one knob, then the second polypeptide comprises a domain including at least one hole on a region other than the second antigen-binding site, wherein, if the first polypeptide comprises at least one hole, then the second polypeptide comprises a domain including at least one knob on a region other than the second antigen-binding site, wherein the knob and the hole can bind to each other so that the first and second polypeptides form dimers, wherein a first tag and a second tag are bound at both termini of the linker, wherein the first tag is linked to the C-terminus of the first polypeptide, the second tag is linked to the N-terminus of the second polypeptide, and the first tag and the second tag each include a cleavable amino acid sequence. Alternatively, the polynuclotide encodes a protein complex including a first polypeptide including a first antigen-binding site at the N-terminus thereof; a second polypeptide including a second antigen-binding site at the N-terminus thereof; and a linker that links the first and second polypeptides to each other, wherein the first and second polypeptides include a domain including at least one knob or hole on a region other than the first or second antigen-binding site, wherein, if the first polypeptide comprises at least one knob, then the second polypeptide comprises a domain including at least one hole on a region other than the second antigen-binding site, wherein, if the first polypeptide comprises at least one hole, then the second polypeptide comprises a domain including at least one knob on a region other than the second antigen-binding site, wherin the knob and the hole bind to each other so that the first and second polypeptides may form dimers, wherein a tag is bound at a terminus of the linker, wherein the tag is linked to the C-terminus of the first polypeptide or the N-terminus of the second polypeptide and includes a cleavable amino acid sequence.

The term “polynucleotide” used herein refers to a polymer of deoxyribonucleotide or ribonucleotide that exists as a single-stranded or double-stranded form. The polynucleotide includes RNA genome sequences, DNA (gDNA and cDNA), or RNA sequences transcribed therefrom, and includes analogues of natural polynucleotides, unless specifically mentioned.

The polynucleotide also includes nucleotide sequences encoding the amino acid sequences of the protein complex or nucleotide sequences complementary thereto. The complementary sequences include completely complementary sequences or substantially complementary sequences. For example, substantially complementary sequences are sequences that may be hybridized with nucleotide sequences encoding the amino acid sequences of the protein complex in stringent conditions known in the art. In particular, substantially complementary sequences are nucleotide sequences that bind to nucleotide sequences with at least 90%, at least 95%, at least 96%, at least 97%, at least 98%, at least 99% identity, or 100% identity to nucleic acid sequence encoding the protein complex.

In addition, the nucleotide sequences encoding the amino acid sequence of the protein complex may be mutated. The mutations include addition, deletion, or non-conservative or conservative substitution of nucleotides. A polynucleotide encoding the amino acid sequence of the protein complex is understood to include nucleotide sequences substantially identical to the nucleotide sequences described above. The substantially identical sequences may be sequences with at least 80% homology/identity, at least 90% homology/identity, or at least 95% homology/identity (e.g., at least 96% identity, at least 97% identity, at least 98% identity, or at least 99% identity) to the nucleotide sequences, when the nucleotide sequences are aligned to correspond to each other as much as possible and the aligned nucleotide sequences are analyzed using an algorithm known in the art.

For example, the polynucleotide may be selected from the group consisting of polynucleotides comprising the nucleic acid sequences of SEQ ID NO: 4 to SEQ ID NO: 6.

According to another embodiment of the present invention, there is provided a recombinant vector that comprises the polynucleotide encoding the protein complex according to an aspect of the present invention and a promoter that is operatively linked to the polynucleotide.

The term “vector” used herein refers to a means of expressing a target gene in a host cell. For example, the vector may be a plasmid vector, a cosmid vector, or a viral vector, such as a bacteriophage vector, an adenovirus vector, a retrovirus vector, or an adeno-associated virus vector. The recombinant vector may be prepared by manipulating a plasmid (for example, pSC101, pGV1106, pACYC177, ColE1, pKT230, pME290, pBR322, pUC8/9, pUC6, pBD9, pHC79, pIJ61, pLAFR1, pHV14, pGEX series, pET series, or pUC19), a phage (for example, λgt4λB, λ-Charon, λΔz1, or M13), or a virus (for example, SV40) as known in the art.

In the recombinant vector, the polynucleotides encoding the protein complex may be operatively linked to a promoter. The term “operatively linked” used herein means a functional linkage between a nucleotide expression regulating sequence (for example, a promoter sequence) and other nucleotide sequences. Thus, the nucleotide expression regulating sequence may regulate the transcription and/or translation of the other nucleotide sequences.

The recombinant vector may be constructed for cloning or expression. For example, a vector for expression may be a vector known in the art for expressing a foreign protein in a plant, animal, or microorganism. The recombinant vector may be constructed using various methods known in the art.

The recombinant vector may be constructed for use in prokaryotic or eukaryotic host cells. For example, when a prokaryotic cell is used as the host cell, the expression vector used generally includes a strong promoter capable of initiating transcription (for example, p_{L}^{λ} promoter, a CMV promoter, trp promoter, lac promoter, tac promoter, or T7 promoter), a ribosome binding site for initiating translation, and a transcription/translation termination sequence. When a eukaryotic cell is used as the host cell, the vector may include an origin of replication acting in the eukaryotic cell, for example, f1 origin of replication, SV40 origin of replication, pMB1 origin of replication, adeno origin of replication, AAV origin of replication, CMV origin of replication or BBV origin of replication, but is not limited thereto. A promoter in an expression vector for a eukaryotic host cell may be a promoter derived from a mammalian genome (for example, a metallothionein promoter) or a promoter derived from a mammalian virus (for example, an adenovirus late promoter, a vaccinia virus 7.5K promoter, an SV40 promoter, a cytomegalovirus (CMV) promoter, or a tk promoter of HSV). A transcription termination sequence in an expression vector for a eukaryotic host cell may be, in general, a polyadenylation sequence.

According to another embodiment of the present invention, there is provided a host cell that includes the recombinant vector.

The host cell, which is capable of stably and consecutively cloning or expressing the recombinant vector, may be any host cell known in the art. A prokaryotic host cell may be, for example, an Escherichia genus bacterium, such as E. coli JM109, E. coli BL21, E. coli RR1, E. coli LE392, E. coli B, E. coli X 1776, or E. coli W3110, a Bacillus genus bacterium, such as Bacillus subtilis, or Bacillus thuringiensis, or an intestinal bacterium, such as Salmonella typhimurium, Serratia marcescens, or various Pseudomonas species. A eukaryotic host cell may be, for example, a yeast (e.g., Saccharomyce cerevisiae), an insect cell, a plant cell, or an animal cell, for example, Sp2/0, Chinese hamster ovary (CHO) K1, CHO DG44, PER.C6, W138, BHK, COS-7, 293, HepG2, Huh7, 3T3, RIN, or an MDCK cell line.

The polynucleotide or the recombinant vector including the same may be transferred into the host cell using a method known in the art. For example, when a prokaryotic cell is used as a host cell, the transfer may be performed using a CaCl₂ method or an electroporation method, and when a eukaryotic cell is used as a host cell, the transfer may be performed by microinjection, calcium phosphate precipitation, electroporation, liposome-mediated transfection, or gene bombardment, but is not limited thereto.

The transformed host cell may be selected using a phenotype expressed by a selectable marker by a method known in the art. For example, when the selectable marker is a specific antibiotic resistance gene, a transformant is cultured in a medium including the antibiotic, and thus, a transformant may easily be selected.

According to another embodiment of the present invention, provided is a method of producing a bi-specific antibody including expressing the recombinant vector to produce the protein complex.

Production of the bi-specific antibody may be performed in vivo or in vitro.

In the case of in vivo production of the bi-specific antibody, a protein complex produced by expressing the recombinant vector in a cell may be released to the outside of the cell in the form of a complete bi-specific antibody. In other words, the protein complex may be produced as a bi-specific antibody such that translation occurs in the endoplasmic reticulum, and then the first and second polypeptides adjacent to each other join together to spontaneously form dimers. Subsequently, the cleavable amino acid sequence of the tag included in the protein complex is cleaved by a protease present in the cell, and, as a result, a bi-specific antibody in a complete form is produced. Here, a production rate of the bi-specific antibody may be increased due to one or more amino acid sequences that bind to each other at the region other than the first and second antigen-binding sites of the first and second polypeptides. Then, the produced bi-specific antibody may be used in a purified form, and the purification method is known in the art.

In the case of in vitro production of the bi-specific antibody, the method may further include cleaving the tag after the expression of the recombinant vector in a cell to produce the protein complex.

The protein complex in vitro is present such that the first and second polypeptides are linked to each other via a linker, and the first and second polypeptides adjacent to each other join together to spontaneously form dimers. Here, one or more amino acid sequences bind to each other at the region other than the first and second antigen-binding sites of the first and second polypeptides, thus a production rate of the bi-specific antibody increases.

In an embodiment, the cleaving process may be performed by adding a protease recognizing the cleavable amino acid sequence included in the tag of the protein complex. In addition, the tag may be selected from, but is not limited to, the group consisting of ubiquitin, ubiquitin-like protein, a TEV cleavage peptide, and a furin cleavage peptide. For example, a protease capable of cleaving the TEV cleavage peptide or the furin cleavage peptide may be added to the protein complex, and, since the TEV cleavage peptide or the furin cleavage peptide is cleaved by the protease, a multi-specific antibody or a bi-specific antibody may be produced from the protein complex.

One or more embodiments of the present invention will now be described more fully with reference to the following examples. However, these examples are for illustrative purposes only and are not intended to limit the scope of the present invention.

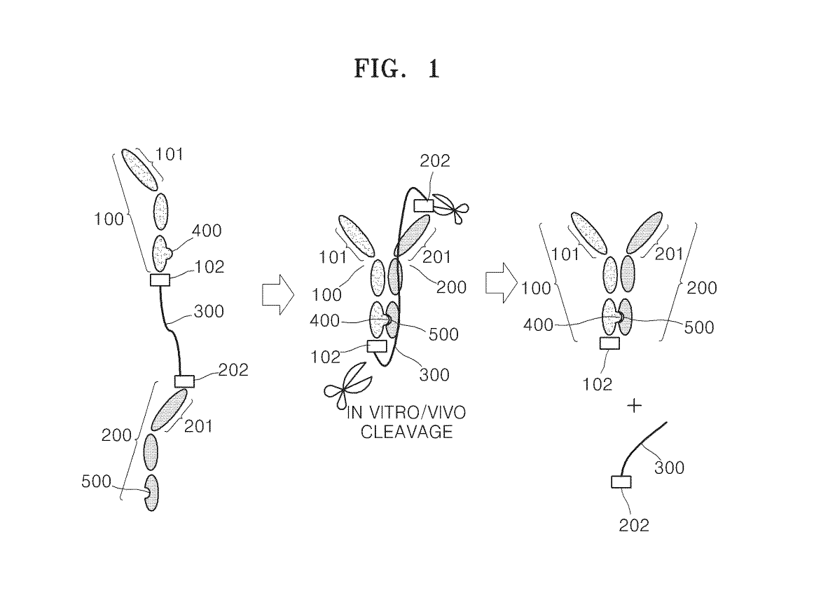

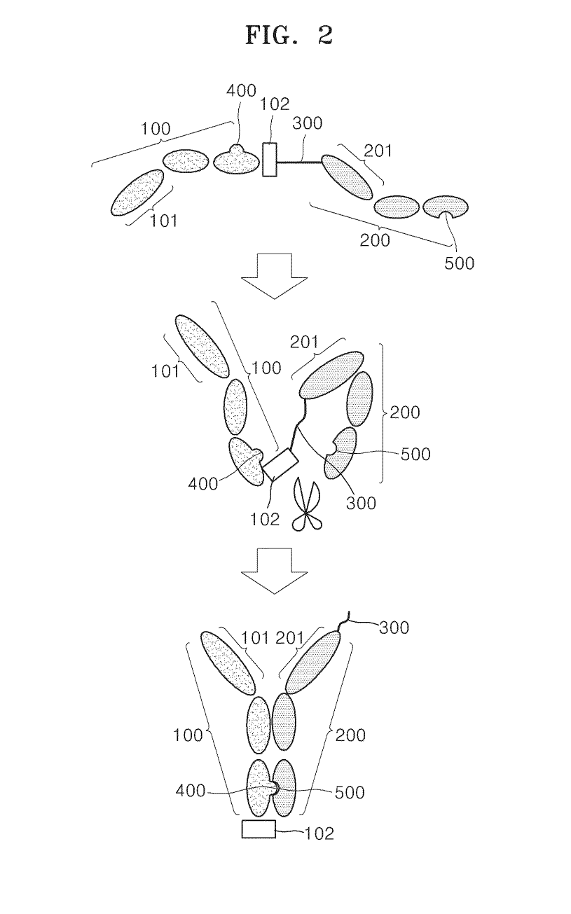

FIGS. 1 and 2 are schematic diagrams illustrating a protein complex including a first peptide 100 including a first antigen-binding site 101 and a second peptide 200 including a second antigen-binding site 201. Referring to FIG. 1, the first polypeptide 100 including the first antigen binding site 101 includes a first tag 102 linked to the terminus thereof, and the second polypeptide 200 including the second antigen binding site 201 includes a second tag 202 linked to the terminus thereof. The first tag 102 and the second tag 202 are respectively linked to the termini of a linker 300 composed of polypeptides. The first tag 102 and the second tag 202 each may be a protein such as ubiquitin or ubiquitin-like protein, and may be subjected to in vitro or in vivo cleavage. The first polypeptide 100 including a first antigen binding site 101 and the second polypeptide 200 including a second antigen binding site 201 are adjacent to each other thus may be joined together in vitro or in vivo via complete, spontaneous binding. Here, a knob 400, which is formed by an amino acid sequence present at a region other than the first antigen-binding site 101 of the first polypeptide 100, and a hole 500, which is formed by an amino acid sequence present at a region other than the second antigen-binding site 201 of the second polypeptide 200, are bound to each other, and thus a production rate of forming a bi-specific protein complex having different antigen binding sites may be increased.

FIG. 2 illustrates a protein complex including the first and second polypeptides 100 and 200 each respectively including the first and second antigen binding sites 101 and 201 illustrated in FIG. 1, in which the second tag 202 is not included. As described above, a bi-specific protein complex having different antigen binding sites is formed through in vitro or in vivo cleavage of the protein complex. In this embodiment, however, the protein complex of FIG. 2 does not include the second tag 202, and thus the protein complex is present in the form such that the linker 300 is linked to the second polypeptide 200 including a second antigen binding site 201. In this regard, the linker 300 includes short amino acid sequences of 2 to 50, and thus does not affect a function of the second polypeptide 200 including a second antigen binding site 201.

### EXAMPLES

### Example 1

### Construction of Expression Vector for the Protein Complex Including Two Antigen Binding Sites

To produce a protein complex of a bi-specific antibody which includes binding sites that are respectively specific to a vascular endothelial growth factor (VEGF) and an epidermal growth factor receptor (EGFR), an expression vector of the protein complex manufactured by Genotech by request was used, and pCDNA 3.1 myc/his A (Invitrogen) was used as a vector for protein overexpression.

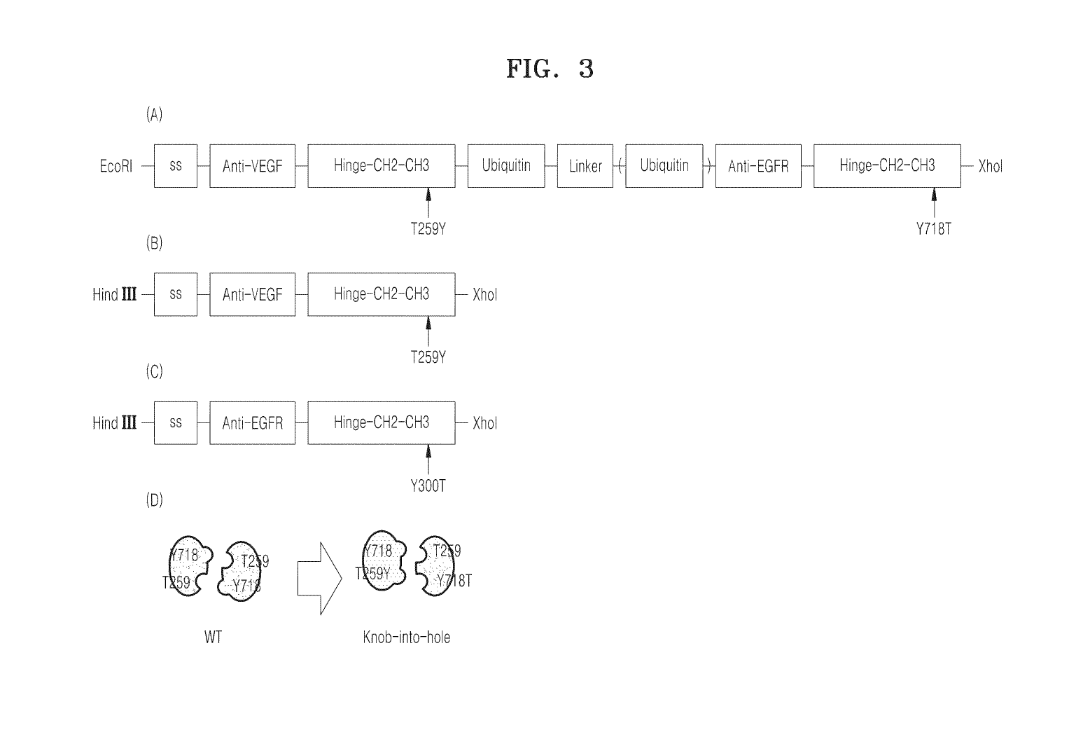

In particular, as illustrated in FIG. 3A, three types of single-sequence DNA corresponding to amino acid sequences of a protein complex that consists of (1) a single-chain polypeptide including a signal sequence (ss), a VEGF-binding site, i.e., V2, and a hinge and consisting of a Fc domain that includes an amino acid sequence forming a knob; (2) a single-chain polypeptide including a EGFR-binding site, i.e., E2 and consisting of a Fc domain that includes an amino acid sequence forming a hole; and (3) at least one ubiquitin tag and a linker were synthesized. To express the protein complex, nucleotide sequences of the three types of single sequence DNA inserted into a plasmid were represented by SEQ ID NOs: 4 to 6.

The inserted DNA fragment includes a nucleotide sequence which is digested with EcoRI at the 5′ terminus thereof and a nucleotide sequence which is digested with Xhol at the 3′ terminus thereof. Thus, the DNA fragment may be inserted into the EcoRI-Xhol restriction site of the vector pcDNA3.1 myc/his A.

Also, two types of DNA were synthesized as follows to compare a bi-specific antibody prepared bythe protein complex and a bi-specific antibody produced by each of the single-chain polypeptides.

As illustrated in FIG. 3B, one type of DNA (SEQ ID NO: 7) corresponding to the amino acid sequence of a single-chain polypeptide consisting of a signal sequence (ss), a VEGF-binding site, i.e., V2, and a hinge and consisting of a Fc domain that includes an amino acid sequence forming a knob and an ubiqitin tag was synthesized and inserted into the vector pCEP4 through the HindIII-Xhol restriction site. Also, as illustrated in FIG. 3C, another type of DNA (SEQ ID NO: 8) corresponding to an amino acid sequence of a single-chain polypeptide consisting of a signal sequence (ss) and a EGFR-binding site, i.e., E2, and consisting of a Fc domain that includes an amino acid sequence forming a hole was synthesized and inserted into the vector pCEP4 through the HindIII-Xhol restriction site.

### Example 2

### Expression of the Protein Complex and Purification of Bi-Specific Antibody

To overexpress a protein complex by using the vector constructed according to Example 1, Human embryonic kidney cells (HEK293-F, available from Korean Cell Line Bank) that were transformed with the vector were used. HEK293-F cells were maintained in an orbital shaker at 37° C. and 130 rpm under 8% CO₂ conditions. To transform the HEK293-F cells, first, the HEK293-F cells were separated from a medium by centrifugation. 1×10⁶ of the HEK293-F cells were suspended in Freestyle 293 Expression media (Invitrogen), and then transformed with 100 μg of the vector by using a FreeStyle™ MAX reagent (Invitrogen). 7 to 8 days after the transformation, the resultant cells were centrifuged (4000× g, 10 min, 4° C.), and a supernatant was collected therefrom and filtered using a filter having a pore size of 0.22 microns. The obtained supernatant was used to purify a bi-specific antibody.

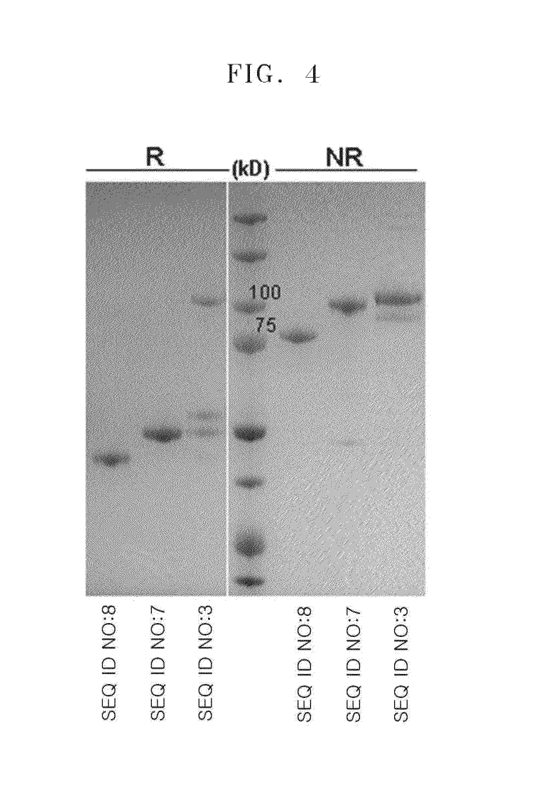

The bi-specific antibody was isolated using a Protein A affinity column (GE Healthcare). First, the Protein A affinity column was equilibrated with 1X PBS (Invitrogen), the supernatant was applied to the equilibrated Protein A affinity column, the resultant column was washed using a washing buffer (1X PBS) having a volume that is five times that of the column, and then the bi-specific antibody was eluted using an IgG elution buffer (Thermo Scientific) containing 10% glycerol. The eluted solution was immediately neutralized with 1 M Tris-HCl (pH 9.0). The eluted solution was converted to 1× PBS through repeatedly centrifugation using an Amicon Ultra-15 Centrifugal Filter (Milipore). A concentration of the purified protein was measured using a Herceptin antibody as a reference material. Thereafter, the concentrated bi-specific antibody was identified by SDS-PAGE. The concentrated bi-specific antibody was divided into two groups, one of which was treated with 1 mM of β-mercaptoethanol (reduction condition: R) and the other of which was not treated with β-mercaptoethanol (non-reduction condition: NR), and they were then loaded on the gel. As a result, as illustrated in FIG. 4, formation of disulfide bonds as a unique property of antibodies was confirmed through the comparison under R and NR conditions, and a homodimeric antibody was not observed.

### Example 3

### Analysis of the Ratio of Bi-Specific Antibody Produced From Protein Complex

A mass analysis was performed to analyze a ratio of heterodimers in the purified protein complex produced according to Example 2. The mass analysis was performed using a high pressure liquid chromatography (HPLC) and a LTQ orbitrap MS system (Thermo Sienctific). A Presto FF-C18 column (Imtakt) was connected to a LC system, and 20 mg of the protein complex was loaded in the column at a temperature of 37° C. and a flow rate of 150 ml/min. A 0.1% trifluoroacetic acid solution with a solvent of water was used as a buffer A, and a 0.1% trifluoroacetic acid solution with a solvent of acetonitrile was used as a buffer B. A protein was separated by increasing a ratio of the buffer B in a mixed solution (the buffer A+the buffer B) from about 3% to about 70% for 32 minutes. Then, the separated protein was introduced to the LTQ orbitrap MS system, and the mass of the protein complex was analyzed. The results are illustrated in Table 1 below.

TABLE 1

| Antibody | Ratio of heterodimers (%) |
|---|---|
| SEQ ID NO: 1 (V2E2-GS30 | 90.47 |
| (KiH)) | |
| SEQ ID NO: 2 (V2E2-2 (KiH)) | 95.88 |
| SEQ ID NO: 3 (V2E2-GS30 + 5 | 96.92 |
| (KiH)) | |

### Example 4

### Confirmation of a Bi-Specific Antigen-Antibody Reaction of Bi-Specific Antibody Produced From the Protein Complex

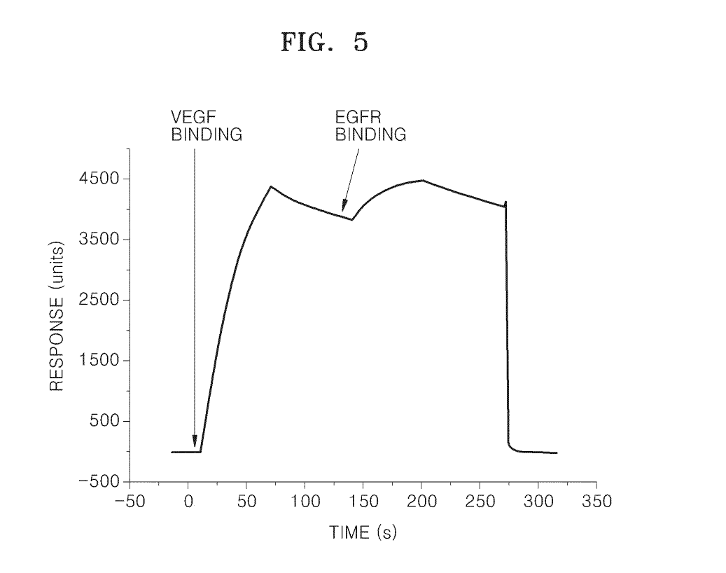

To measure a binding affinity of a bi-specific antigen-antibody reaction of the bi-specific antibody produced according to Example 2, a surface plasmon resonance test was performed using a BiacoreT100 instrument (GE healthcare). 1X HBS-EP (GE healthcare) was used as a running buffer and a dilution buffer. About 5,000 response unit (RU) of an Anti-human IgG antibody (Jackson Immuno Research) were immobilized on a surface of a CM5 chip (GE healthcare) by standard amine-coupling. About 500 RU of the bi-specific antibody was added to the CM5 chip so as to bind thereto, and then several concentrations (about 6.25 to about 100 nM) of human EGFR extracellular domain (Prospec) or human VEGF (pangen) were added to the CM5 chip at a flow rate of about 50 μL/min. A contact time (association phase) was about 180 seconds, and a separation time (washing with running buffer) was about 600 seconds. After each binding cycle was terminated, Glycine-HCl pH 2.0 (GE healthcare) as a regeneration solution was added to the chip at a flow rate of about 50 μL/min for about 1 minute to remove the combined antigen and antibody from the chip. A sensorgram was obtained therefrom such that a fitting process was performed in Biospecific Interaction Analysis (BIA) evaluation software by using a 1:1 Langmuir binding model for the EGFR case and by using a bivalent analyte model for the VEGF case. The results are illustrated in FIG. 5.

As described above, according to the one or more of the above embodiments of the present invention, by using a protein complex, a system that simultaneously targets two antigens may be effectively constructed.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A protein complex comprising:
a first polypeptide comprising a first antigen-binding site at the N-terminus thereof;
a second polypeptide comprising a second antigen-binding site at the N-terminus thereof; and
a linker that links the first and second polypeptides to each other,
wherein the first polypeptide and second polypeptide each comprises a domain including at least one knob or hole on a region other than the first or second antigen-binding site,
wherein, if the first polypeptide comprises at least one knob, then the second polypeptide comprises a domain including at least one hole on a region other than the second antigen-binding site,
wherein, if the first polypeptide comprises at least one hole, then the second polypeptide comprises a domain including at least one knob on a region other than the second antigen-binding site,
wherein the knob and the hole bind to each other so that the first and second polypeptides form dimers,
wherein a tag is bound to a terminus of the linker, and
wherein the tag is linked to the C-terminus of the first polypeptide or the N-terminus of the second polypeptide and comprises a cleavable amino acid sequence.

2. The protein complex of claim 1, wherein a first tag and a second tag are bound at both termini of the linker, and
wherein the first tag is linked to the C-terminus of the first polypeptide, the second tag is linked to the N-terminus of the second polypeptide, and the first tag and the second tag each comprise a cleavable amino acid sequence.

3. The protein complex of claim 1, wherein the first and second antigen-binding sites are antigen-binding sites that are identical to or different from each other.

4. The protein complex of claim 1, wherein the region other than the first and second antigen-binding sites is a CH3 domain of a Fc region of an antibody.

5. The protein complex of claim 1, wherein the first polypeptide is a polypeptide comprising an antigen-binding site selected from the group consisting of a single-domain antibody, Fab, Fab′, and scFv, and a Fc domain.

6. The protein complex of claim 1, wherein the second polypeptide is a polypeptide comprising an antigen-binding site selected from the group consisting of a single-domain antibody, Fab, Fab′, and scFv, and a Fc domain.

7. The protein complex of claim 1, wherein the tag is selected from the group consisting of ubiquitin, ubiquitin-like protein, a TEV cleavage peptide, and a furin cleavage peptide.

8. The protein complex of claim 1, wherein the linker is a polypeptide of 1 to 100 amino acids.

9. The protein complex of claim 1, wherein the protein complex comprises the amino acid sequence of SEQ ID NO: 1 or SEQ ID NO: 2.

10. The protein complex of claim 1, wherein the protein complex comprises the amino acid sequence of SEQ ID NO: 3.

11. A polynucleotide encoding the protein complex of claim 1.

12. The polynucleotide of claim 11, wherein the polynucleotide comprises a nucleic acid sequence selected from the group consisting of SEQ ID NO: 4 to SEQ ID NO: 6.

13. A recombinant vector comprising the polynucleotide of claim 11 and a promoter that is operatively linked to the polynucleotide.

14. A host cell comprising the recombinant vector of claim 13.

15. A method of preparing a bi-specific antibody, the method comprising expressing the recombinant vector of claim 13 to produce the protein complex, thereby preparing a bi-specific antibody.

16. The method of claim 15, further comprising cleaving the tag after expression of the recombinant vector to produce the protein complex.

17. The method of claim 16, wherein the cleaving is performed by adding a protease that recognizes a cleavable amino acid sequence of the tag of the protein complex.

18. The protein complex of claim 2, wherein the first polypeptide is a polypeptide comprising an antigen-binding site selected from the group consisting of a single-domain antibody, Fab, Fab′, and scFv, and a Fc domain.

19. The protein complex of claim 2, wherein the second polypeptide is a polypeptide comprising an antigen-binding site selected from the group consisting of a single-domain antibody, Fab, Fab′, and scFv, and a Fc domain.

20. The protein complex of claim 2, wherein the tag is selected from the group consisting of ubiquitin, ubiquitin-like protein, a TEV cleavage peptide, and a furin cleavage peptide.
